SEQUENCE LISTING (3 sequences)

SEQ ID NO: 1 - DNA, 20 bp, Artificial Sequence
  TRAC targeting gRNA
agagtctctcagctggtaca

SEQ ID NO: 2 - DNA, 20 bp, Artificial Sequence
  CD7 targeting gRNA
ggagcaggtgatgttgacgg

SEQ ID NO: 3 - DNA, 20 bp, Artificial Sequence
  beta 2 microglobulim guide RNA
ggccgagatgtctcgctccg

(19) United States
(12) Patent Application Publication
(10) Pub. No.: US 2024/0166995 A1
(43) Pub. Date: May 23, 2024

(54) SEQUENTIAL DELIVERY OF RNPS TO IMMUNE CELLS

(71) Applicant: Avectas Limited, Co. Kildare (IE)

(72) Inventors: Shirley O'Dea, Maynooth (IE); Michael Maguire, Dublin (IE)

(21) Appl. No.: 18/272,699

(22) PCT Filed: Jan. 20, 2022
(86) PCT No.: PCT/IB22/50492

§ 371 Date: Jul. 17, 2023

Related U.S. Application Data
(60) Provisional application No. 63/139,649, filed on Jan. 20, 2021.

(51) Int. Cl. C12N 5/0783; C07K 14/705; C07K 14/725; C12N 15/113; C12N 15/87

(52) U.S. Cl. CPC C12N 5/0636; C07K 14/70503; C07K 14/7051; C12N 15/1138; C12N 15/87; C12N 2310/20; C12N 2320/32; C12N 2500/60; C12N 2510/00

(57) ABSTRACT

The invention provides a solution to the problem of transfecting non-adherent cells. Methods and compositions containing ethanol and an isotonic salt solution are used for delivery of compounds and compositions to mammalian cells.

### RELATED APPLICATIONS

This application claims the benefit of priority under 35 U.S.C. § 119(e) to U.S. Provisional Application No. 63/139,649, filed Jan. 20, 2021, the entire contents of which is incorporated herein by reference in its entireties.

### INCORPORATION BY REFERENCE OF SEQUENCE LISTING

The contents of the sequence listing text file named “048831-528001WO_Sequence_Listing_ST25.txt”, which was created on Jan. 20, 2022 and is 4096 bytes in size, is hereby incorporated by reference in its entirety.

### FIELD OF THE INVENTION

The invention relates to the delivery of agents into mammalian cells for immunotherapy.

### BACKGROUND

Variability in cell transfection efficiency exists among different cell types. Transfection of suspension cells, e.g., non-adherent cells, has proven to be difficult using conventional methods. Thus, a need exists for compositions and methods to facilitate transfection of such cells.

### SUMMARY OF THE INVENTION

The invention provides a solution to engineering immune cells for ex vivo cell therapy applications. The compositions and methods described herein facilitate cell engineering technologies that enable next generation cell therapy products which require complex modifications and high levels of cell functionality. As described herein, the SOLUPORE® delivery method is a non-viral means of simply, rapidly and efficiently delivering cargos to primary immune cells, while retaining cell viability and functionality. The method comprises alcohol-based transient permeabilization of the cell membrane of the processed cells. Moreover, cells engineered in this manner, e.g., engineered immune cells such as T-cells, reduce likelihood of T cell exhaustion, thus enabling their use for complex therapeutic needs.

In aspects, provided herein is an immune cell comprising at least two exogenous cargos. For example, the exogenous cargo comprises a T cell receptor alpha constant (TRAC) ribonucleoprotein (RNP) or a cluster of differentiation 7 (CD7) RNP.

In embodiments, the exogenous cargos are sequentially delivered.

In further examples, the viability of the immune cell is increased compared to a control immune cell.

Also provided herein are methods for delivering at least two exogenous cargos across a plasma membrane of a non-adherent immune cell, where the method includes providing a population of non-adherent cells; and contacting the population of cells with a volume of an isotonic aqueous solution, the aqueous solution including the exogenous cargo and an alcohol at greater than 0.2 percent (v/v) concentration, e.g., 2-20% (v/v) ethanol. In embodiments, the at least two exogenous cargos are sequentially delivered. In other examples, the at least two exogenous cargos include a ribonucleoprotein (RNP), a nucleic acid, a protein, or any combination thereof.

For example, the exogenous cargo comprises a T cell receptor alpha constant (TRAC) ribonucleoprotein (RNP) or a cluster of differentiation 7 (CD7) RNP.

The exogenous cargo, or “payload” are terms used to describe a compound, e.g., an RNP, or composition that is delivered via an aqueous solution across a cell plasma membrane and into the interior of a cell.

The term, “exogenous” refers to cargo (or payload) coming from or deriving from outside the cell, e.g., an immune cell, as opposed to an endogenous agent that originated within the immune cell.

In some embodiments, the immune cell of the invention comprises at least two or more exogenous cargos (e.g., 3, 4, 5, 6 7, 8, 9, or 10 exogenous cargos). The exogenous cargo comprises a ribonucleoprotein (RNP), a nucleic acid, a protein, or any combination thereof.

Also provided herein is a method of delivering at least two exogenous cargos (or an “exogenous cargo”) across a plasma membrane of a non-adherent immune cell. Accordingly, the method includes providing a population of non-adherent cells and contacting the population of cells with a volume of an isotonic aqueous solution, the aqueous solution including the payload and an alcohol at greater than 0.2 percent (v/v) concentration.

For example, the alcohol concentration is about 0.2 percent (v/v) concentration or greater, or the alcohol is about 0.5 percent (v/v) concentration or greater, or the alcohol is about 2 percent (v/v) or greater concentration. Alternatively, the alcohol concentration 5 percent (v/v) or greater. In other examples, the alcohol is 10 percent (v/v) or greater concentration.

For example, the alcohol comprises ethanol, e.g., 10% ethanol or greater. In some examples, the aqueous solution comprises between 20-30% ethanol, e.g., 27% ethanol. In other examples, delivery of an RNP comprises 10% ethanol.

In examples, the aqueous solution includes alcohol, and the alcohol may include ethanol. In other examples, the aqueous solution comprises greater than 10% ethanol, between 20-30% ethanol, or about 27% ethanol. In examples, the aqueous solution comprises between 12.5-500 mM potassium chloride (KCl), or about 106 mM KCl.

The aqueous solution for delivering the exogenous cargo to cells comprises a salt, e.g., potassium chloride (KCl) in between 12.5-500 mM. For example, the solution is isotonic with respect to the cytoplasm of a mammalian cell such as a human T cell. Such an exemplary isotonic delivery solution 106 mM KCl.

In other examples, the aqueous solution can include an ethanol concentration of 5 to 30% (e.g., 0.2% to 30%). The aqueous solution can include one or more of 75 to 98% H₂O, 2 to 45% ethanol, 6 to 91 mM sucrose, 2 to 500 mM KCl, 2 to 35 mM ammonium acetate, and 1 to 14 mM (4-(2-hydroxyethyl)-1-piperazineethanesulfonic acid) (HEPES). For example, the delivery solution contains 106 mM KCl and 27% ethanol. For example, the delivery solution contains 106 mM KCl and 10% ethanol. For example, the delivery solution contains 106 mM KCl and 5% ethanol. For example, the delivery solution contains 106 mM KCl and 2% ethanol.

Exemplary non-adherent/suspension cells include primary hematopoietic stem cell (HSC), T cells (e.g., CD3+ cells, CD4+ cells, CD8+ cells), natural killer (NK) cells, cytokine-induced killer (CIK) cells, human cord blood CD34+ cells, B cells, or cell lines such as Jurkat T cell line. The non-adherent cells can be substantially confluent, such as greater than 75 percent confluent. Confluency of cells refers to cells in contact with one another on a surface. For example, it can be expressed as an estimated (or counted) percentage, e.g., 10% confluency means that 10% of the surface, e.g., of a tissue culture vessel, is covered with cells, 100% means that it is entirely covered. For example non-adherent cells can be spun down, pulled down by a vacuum, or tissue culture medium aspiration off the top of the cell population, or removed by aspiration or vacuum removal from the bottom of the vessel. The cells can form a monolayer of cells. For example, for the cells are 10, 25, 50, 75, 90, 95, or 100% confluent.

In embodiments, the non-adherent cell comprises a peripheral blood mononuclear cell. In examples, the non-adherent cell comprises an immune cell, for example a T lymphocyte.

The method involves delivering the exogenous cargo (e.g., at least two exogenous cargos) in the delivery solution to a population of non-adherent cells comprising a monolayer. For example, the monolayer is contacted with a spray of aqueous delivery solution. The method delivers the payload/cargo (compound or composition) into the cytoplasm of the cell and wherein the population of cells comprises a greater percent viability compared to delivery of the payload by electroporation or nucleofection, a significant advantage of the Soluporation system.

In certain embodiments, the monolayer of non-adherent/suspension cells resides on a membrane filter. In some embodiments, the membrane filter is vibrated following contacting the cell monolayer with a spray of the delivery solution. The membrane filter may be vibrated or agitated before, during, and/or after spraying the cells with the delivery solution.

The volume of solution to be delivered to the cells is a plurality of units, e.g., a spray, e.g., a plurality of droplets on aqueous particles. The volume is described relative to an individual cell or relative to the exposed surface area of a confluent or substantially confluent (e.g., at least 75%, at least 80% confluent, e.g., 85%, 90%, 95%, 97%, 98%, 100%) cell population. For example, the volume can be between 6.0×10⁻⁷ microliter per cell and 7.4×10⁻⁴ microliter per cell. The volume is between 4.9×10⁻⁶ microliter per cell and 2.2×10⁻³ microliter per cell. The volume can be between 9.3×10⁻⁶ microliter per cell and 2.8×10⁻⁵ microliter per cell. The volume can be about 1.9×10⁻⁵ microliters per cell, and about is within 10 percent. The volume is between 6.0×10⁻⁷ microliter per cell and 2.2×10⁻³ microliter per cell. The volume can be between 2.6×10⁻⁹ microliter per square micrometer of exposed surface area and 1.1×10⁻⁶ microliter per square micrometer of exposed surface area. The volume can be between 5.3×10⁻⁸ microliter per square micrometer of exposed surface area and 1.6×10⁻⁷ microliter per square micrometer of exposed surface area. The volume can be about 1.1×10⁻⁷ microliter per square micrometer of exposed surface area.

Throughout the specification the term “about” can be within 10% of the provided amount or other metric.

Confluency of cells refers to cells in contact with one another on a surface. For example, it can be expressed as an estimated (or counted) percentage, e.g., 10% confluency means that 10% of the surface, e.g., of a tissue culture vessel, is covered with cells, 100% means that it is entirely covered. For example, adherent cells grow two dimensionally on the surface of a tissue culture well, plate or flask. Non-adherent cells can be spun down, pulled down by a vacuum, or tissue culture medium aspiration off the top of the cell population, or removed by aspiration or vacuum removal from the bottom of the vessel.

The payload (exogenous cargo) can include a small chemical molecule, a peptide or protein, or a nucleic acid. The small chemical molecule can be less than 1,000 Da. The chemical molecule can include MitoTrackerg Red CMXRos, propidium iodide, methotrexate, and/or DAPI (4′,6-diamidino-2-phenylindole). The peptide can be about 5,000 Da. The peptide can include ecallantide under trade name Kalbitor, is a 60 amino acid polypeptide for the treatment of hereditary angioedema and in prevention of blood loss in cardiothoracic surgery), Liraglutide (marketed as the brand name Victoza, is used for the treatment of type II diabetes, and Saxenda or the treatment of obesity), and Icatibant (trade name Firazyer, a peptidomimetic for the treatment of acute attacks of hereditary angioedema). The small-interfering ribonucleic acid (siRNA) molecule can be about 20-25 base pairs in length, or can be about 10,000-15,000 Da. The siRNA molecule can reduces the expression of any gene product, e.g., knockdown of gene expression of clinically relevant target genes or of model genes, e.g., glyceraldehyde-3 phosphate dehydrogenase (GAPDH) siRNA, GAPDH siRNA-FITC cyclophilin B siRNA, and/or laminsi RNA. Protein therapeutics can include peptides, enzymes, structural proteins, receptors, cellular proteins, or circulating proteins, or fragments thereof. The protein or polypeptide be about 100-500,000 Da, e.g., 1,000-150,000 Da.

The payload (or “exogenous cargo”) can include a therapeutic agent. A therapeutic agent, e.g., a drug, or an active agent, can mean any compound useful for therapeutic or diagnostic purposes, the term can be understood to mean any compound that is administered to a patient for the treatment of a condition. Accordingly, a therapeutic agent can include, proteins, peptides, antibodies, antibody fragments, and small molecules. Therapeutic agents described in U.S. Pat. No. 7,667,004 (incorporated herein by reference) can be used in the methods described herein. The therapeutic agent can include at least one of cisplatin, aspirin, statins (e.g., pitavastatin, atorvastatin, lovastatin, pravastatin, rosuvastatin, simvastatin, promazine IICl, chloropromazine HO, thioridazine HCl, Polymyxin B sulfate, chloroxine, benfluorex HCl and phenazopyridine HCl), and fluoxetine. The payload can include a diagnostic agent. The diagnostic agent can include a detectable label or marker such as at least one of methylene blue, patent blue V, and Indocyanine green. The payload can include a fluorescent molecule. The payload can include a detectable nanoparticle. The nanoparticle can include a quantum dot.

The payload (“exogenous cargo”) includes an alcohol. By the term “an alcohol” is meant a polyatomic organic compound including a hydroxyl (—OH) functional group attached to at least one carbon atom. The alcohol may be a monohydric alcohol and may include at least one carbon atom, for example methanol. The alcohol may include at least two carbon atoms (e.g. ethanol). In other aspects, the alcohol comprises at least three carbons (e.g. isopropyl alcohol). The alcohol may include at least four carbon atoms (e.g., butanol), or at least seven carbon atoms (e.g., benzyl alcohol). The example payload may include no more than 50% (v/v) of the alcohol, more preferably, the payload comprises 2-45% (v/v) of the alcohol, 5-40% of the alcohol, and 10-40% of the alcohol. The payload may include 20-30% (v/v) of the alcohol.

Most preferably, the payload delivery solution includes 25% (v/v) of the alcohol. Alternatively, the payload can include 2-8% (v/v) of the alcohol, or 2% of the alcohol. The alcohol may include ethanol and the payload comprises 5, 10, 20, 25, 30, and up to 400/0 or 50% (v/v) of ethanol, e.g., 27%. Example methods may include methanol as the alcohol, and the payload may include 5, 10, 20, 25, 30, or 40% (v/v) of the methanol. The payload may include 2-45% (v/v) of methanol, 20-30% (v/v), or 25% (v/v) methanol. Preferably, the payload includes 20-30% (v/v) of methanol. Further alternatively, the alcohol is butanol and the payload comprises 2, 4, or 8% (v/v) of the butanol.

In some aspects of the present subject matter, the payload is in an isotonic solution or buffer.

According to the present subject matter, the payload may include at least one salt. The salt may be selected from NaCl, KCl, Na₂HPO₄, C₂H₃O₂NH₄ and KH₂PO₄. For example, KCl concentration ranges from 2 mM to 500 mM. In some preferred embodiments, the concentration is greater than 100 mM, e.g., 106 mM. According to example methods of the present subject matter, the payload may include a sugar (e.g., a sucrose, or a disaccharide). According to example methods, the payload comprises less than 121 mM sugar, 6-91 mM, or 26-39 mM sugar. Still further, the payload includes 32 mM sugar (e.g., sucrose). Optionally, the sugar is sucrose and the payload comprises 6.4, 12.8, 19.2, 25.6, 32, 64, 76.8, or 89.6 mM sucrose.

In embodiments, the methods for delivering an exogenous cargo across the plasma membrane of the immune cell further comprise delivering at least two exogenous cargos (or “two payloads”). The exogenous cargo comprises a ribonucleoprotein, a nucleic acid, a protein, or any combination thereof. In examples, the immune cells comprises two exogenous cargos, 3 exogenous cargos, 4, 5, 6, 7, 8, 9, or 10 exogenous cargos.

In embodiments, the at least two exogenous cargos are sequentially delivered. For example, sequentially delivered may refer to delivery of one exogenous cargo, followed by delivery of a second, third, or fourth exogenous cargo. For example the immune cell of the invention (comprising an exogenous cargo), may then further be manipulated to comprise a second exogenous cargo. As used herein, the term “manipulated” may refer to any known transfection method for intracellular delivery, including the SOLUPORE® delivery method, membrane-disrupting methods (electroporation, sonoporation, magnetotection, optoperation), or carrier-based methods (lipid nanoparticles). Electroporation, for example, includes an intracellular delivery method where an electrical field is applied to cells to increase the cell membrane permeability (also called electrotransfer).

In other aspects, provided herein is a method of delivering a an exogenous cargo across a plasma membrane of a non-adherent cell, the method comprising the steps of providing a population of non-adherent cells and using at least two intracellular delivery methods selected from (i) contacting the population of cells with a volume of an isotonic aqueous solution, the aqueous solution including the exogenous cargo and an alcohol at greater than 0.5 percent (v/v) concentration. The concentration of alcohol in the aqueous solution is greater than 0.2 percent (v/v) concentration, or greater than 0.5 percent (v/v) concentration, or greater than 2 percent (v/v) concentration, or greater than 5 percent (v/v) concentration, or greater than 10 percent (v/v) concentration. In some examples, the aqueous solution comprises between 20-30% ethanol, e.g., 27% ethanol.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### DESCRIPTION OF THE DRAWINGS

FIGS. 1A-1D are bar graphs showing efficient engineering of T cells with the SOLUPORE® delivery method. FIG. 1A is a graph showing GFP expression and cell viability at 24 hr post-GFP mRNA delivery to T cells from 3 donors. Delivery is represented as the total percentage of cells. FIG. 1B is a line graph showing the projected proliferation curves post-GFP mRNA delivery to T cells from 1 donor, showing mean of n=2. No significant difference in proliferation is observed between SOLUPORE®-treated cells and untreated control cells (Wilcoxon matched pairs signed rank test). FIG. 1C is a graph showing the co-delivery of GFP mRNA and CD19 CAR mRNA, n=3 in 1 donor. FIG. 1D is a graph showing the delivery of TRAC and CD7 RNPs individually and delivery of TRAC RNP followed 2 days later by CD7 RNP with analysis at day 5 post-CD7 RNP delivery, n=3 in 1 donor. (SOL=SOLUPORE® delivery system).

FIGS. 2A and 2B are bar graphs showing that the percentage population that was negative for all 3 proteins was 46±7 and 38±9 from SOLUPORE® Research Platform delivery system and the clinical use platform, respectively (3 donors, 3 technical repeats); FIGS. 2A and 2B, respectively. Comparable performance on the SOLUPORE® Research Platform delivery system and the clinical platform was observed, with 10% or less triple edits by other techniques.

FIG. 3 is a schematic of the experimental design for simultaneous delivery of RNPs. Cas9 RNP—TRAC sgRNA was prepared at 2:1 ratio at 0.4 μg/μL (equiv to 3.3 μg per 1×10⁶ cells); S Buffer solutions were prepared with 0, 5, 10 and 15% ethanol with RNP and the experiments were carried out on the SOLUPORE® delivery system with the S buffer solutions at each ethanol concentration.

FIG. 4 depicts representative flow cytometry plots from cells stained with an antibody targeting CD3 (gated off the live population). Untreated (UT) cells showed >93% positivity for CD3 and this was reduced following delivery of TRAC RNP by the SOLUPORE® Research Platform delivery system. Two distinct populations are observed in the treated samples with the population on the left (gated) referring to those cells that were negative for CD3 staining. This negative population increased from ˜59% in samples where no ethanol was present in the delivery Solution to ˜67% in samples where ethanol was present. A limit exists to the amount of ethanol present before precipitation of the Cas9 protein occurs (>20% ethanol at 0.4 μg/μL Cas9 RNP).

FIG. 5A is a bar graph showing the mean CD3 negative population (±standard deviation) from 2-3 replicates per condition in activated T cells 72 hr post-delivery of TRAC RNP (2:1 guide to Cas9 molar ratio; 3.3 μg per 1×10⁶ cells) by the SOLUPORE® Research Platform delivery system. Increasing concentrations of ethanol were added with the cargo in the delivery solution. The level of CD3 edit increased modestly with increasing concentrations of ethanol (0% EtOH-58% to 15% EtOH-66%).

FIG. 5B is a table showing the mean, standard deviation, standard error of the mean and coefficient of variation of CD3 negative expression from each group 72 hr post-delivery of TRAC RNP by the SOLUPORE® Research Platform delivery system.

FIG. 6 is a bar graph depicting the percent viability at the increasing ethanol concentrations, and time points consisting of pre-delivery, post delivery (day 3) and post delivery (day 5).

FIGS. 7A-7C are line graphs showing CRISPR/RNP editing in Human Primary T Cells. FIG. 7A is a graph showing the CD3 negative dose response using TRAC RNP (μg per 1×10⁶ cells). FIG. 7B is a graph showing the CD7 negative dose response using CD7 RNP (μg per 1×10⁶ cells). FIG. 7C is a graph showing the HLA negative dose response using β-2 microglobulin (B2m) RNP (μg per 1×10⁶ cells). Successful edits for multiple TRAC (CD3), CD7, B2M (HLA) RNPs with >80% viability was observed.

FIG. 8 is a bar graph showing that T cell phenotype was preserved during editing.

### DETAILED DESCRIPTION

Provided herein are, cell engineering technologies that enable next generation cell therapy products which require complex modifications and high levels of cell functionality.

Autologous chimeric antigen receptor (CAR) T cell therapy has shown unprecedented efficacy as well as durable responses in cohorts of relapsed or refractory cancer patients with certain liquid tumors resulting in two CAR T product approvals to date (Ahmad A, Uddin S, Steinho M. CAR-T cell therapies: An overview of clinical studies supporting their approved use against acute lymphoblastic leukemia and large b-cell lymphomas. Int J Mol Sci 2020; 21:3906). The proof of concept generated with these cell products is now driving significant levels of research, development and commercial activity in the ex vivo cell therapy field.

Viral transduction has been the most commonly used method for cell engineering and these first approved CAR T cell therapies were engineered using viral vectors. However, limitations of viral vectors are well-known. Specialized viral manufacturing processes combined with constraints on availability of manufacturing capacity make scaling to meet commercial demand a significant challenge (Levine B, Miskin J, Wonnacott K, Keir C. Global Manufacturing of CAR T Cell Therapy. Mol Ther 2017; 4:92-101). The timeline from initiation of virus production to batch release of GMP vector for cellular therapies can be lengthy and costly. Viral delivery systems are also susceptible to vector-mediated genotoxicity, such as random insertions that disrupt normal genes, accidental oncogene activation or insertional mutagenesis leading to adverse immunogenicity and severe side effects (David R, Doherty A. Viral Vectors: The road to reducing genotoxicity. Toxicol Sci 2017; 155:315-25). In addition, constraints on the cargo packaging capacity of viral vectors are a further limitation on their applicability to the engineering of next generation cell therapy products which will require complex modifications in order to successfully address solid tumors and for allogeneic products (Rafiq S, Hackett C, Brentjens R. Engineering strategies to overcome the current roadblocks in CAR T cell therapy. Nat Rev Clin Oncol 2020; 17:147-67).

The scaling, cost and safety challenges associated with viral vectors have driven an interest in the development of non-viral alternatives. Greater multiplexing potential, flexibility and versatility to accommodate diverse cell types and accelerated manufacturing timelines, all whilst avoiding the manufacturing challenges, side effects and regulatory burden associated with viral vectors, are attractive attributes in any one intracellular delivery method (Stewart M, Sharei A, Ding X, Sahay G, Langer R, Jensen K. In vitro and ex vivo strategies for intracellular delivery. Nature 2016; 538:183-92). Non-viral intracellular delivery methods can be broadly classified into two main categories. The first includes physical/mechanical methods such as electroporation, sonoporation, magnetofection, gene gun, microinjection and cell squeezing (Stewart M, Langer R, Jensen K. Intracellular delivery by membrane disruption: Mechanisms, strategies, and concepts. Chem Rev 2018; 118: 7409-531, Bono N, Ponti F, Mantovani D, Candiani G. Non-viral in vitro gene delivery: It is now time to set the bar. Pharmaceutics 2020; 12:183, and DiTommaso T, et al. Cell engineering with microfluidic squeezing preserves functionality of primary immune cells in vivo. Proc Natl Acad Sci USA 2018; 115: E10907-E10914). However, many of these methods are unsuited to cell therapy manufacturing processes.

The second category includes chemical vectors such as cationic polymers and lipids, including lipid nano-particles but to date, the efficiency of these chemical methods is not comparable to viral counterparts and toxicity remains a concern (Azarnezhad A, Samadian H, Jaymand M, Sobhani M, Ahmadi A. Toxicological profile of lipid-based nanostructures: are they considered as completely safe nanocarriers? Crit Rev Toxicol 2020; 50: DOI: 148-176). As a result, electroporation platforms are currently the most widely used non-viral technologies for cell engineering.

Next generation immune cell products are likely to be both virally and non-virally modified. The first human trial to test the safety of CRISPR-Cas9 gene editing of T cells employed electroporation to deliver the gene editing tools prior to viral transduction to deliver the CAR (Stadtmauer E, et. al., CRISPR-engineered T cells in patients with refractory cancer. Science 2020; 367: eaba7365). However, concerns persist regarding the impact of the electroporation process on immune cells, including sustained intracellular calcium levels, changes in gene expression profiles, reduced proliferative capacity and decreased potency (DiTommaso T et al. Cell engineering with microfluidic squeezing preserves functionality of primary immune cells in vivo. Proc Natl Acad Sci USA 2018; 115: E10907-E10914, Zhang M, et al. The impact of Nucleofection on the activation state of primary human CD4 T cells. J Immunol Methods 2014; 408:123-31, and Beane J, et al. Clinical Scale Zinc Finger Nuclease-mediated Gene Editing of PD-1 in Tumor Infiltrating Lymphocytes for the Treatment of Metastatic Melanoma. Mol Ther 2015; 23:1380-90).

It has become clear that robust immune cell proliferation and effector function in vitro correlate with improved antitumor function in vivo, highlighting the need for delivery methods that do not negatively impact these critical quality attributes of the effector cells (Ghassemi S, et al Reducing ex vivo culture improves the antileukemic activity of chimeric antigen receptor (CAR) T cells. Can Immunol Res 2018; 6:1100-9 and Finney O, et al. CD19 CAR T cell product and disease attributes predict leukemia remission durability. J Clin Invest 2019; 129:2123-32). In addition, to ensure that manufacturing is feasible in terms of cost, time and meeting the urgent clinic need, it is critical to achieve sufficient yields of viable transgene-positive cells to produce a final product to treat patients. Therefore, alternative approaches are required and the ideal intracellular delivery method will allow transfection of a diverse array of cargo to multiple cell types whilst minimally perturbing normal cell function.

A non-viral method using the SOLUPORE® delivery system was reported that allows for transient permeabilization of the cell membrane to achieve rapid intracellular delivery of cargos with varying composition, properties and size such as macromolecules and nucleic acids (O'Dea S, et al. Vector-free intracellular delivery by reversible permeabilization. PLoS ONE 2017; 12). Here the SOLUPORE® delivery system was shown to successfully facilitate the delivery of mRNA and gene-editing tools such as CRISPR-Cas9 to primary human T cells with retention of cell viability.

### Exogenous Cargo

The exogenous cargo (or “payload”) delivered to the immune cell describes a compound, or composition that is delivered via an aqueous solution across a cell plasma membrane and into the interior of a cell. The exogenous cargo can include a nucleic acid (for example, RNA (ribonucleic acid), mRNA (messenger RNA), or DNA (deoxyribonucleic acid)), a protein or peptide, a small chemical molecule, or any combination thereof. The small chemical molecule can be less than 1,000 Da. A small molecule is a compound that is less than 2000 Daltons in mass. The molecular mass of the small molecule is preferably less than 1000 Daltons, more preferably less than 600 Daltons, e.g., the compound is less than 500 Daltons, 400 Daltons, 300 Daltons, 200 Daltons, or 100 Daltons.

In preferred examples, the exogenous cargo comprises a ribonucleoprotein (RNP), e.g., TRAC (T cell receptor alpha constant) RNP or CD7 (cluster of differentiation) RNP.

### Sequential Delivery of Exogenous Cargo

In embodiments, the at least two exogenous cargos are sequentially delivered. For example, sequentially delivered may refer to delivery of one exogenous cargo, followed by delivery of a second, third, or fourth exogenous cargo. For example the immune cell of the invention (comprising an exogenous cargo), may then further be manipulated to comprise a second exogenous cargo. As used herein, the term “manipulated” may refer to any known transfection method for intracellular delivery, including the SOLUPORE® delivery method, membrane-disrupting methods (electroporation, sonoporation, magnetotection, optoperation), or carrier-based methods (lipid nanoparticles).

In preferred examples, a TRAC RNP is delivered, which is followed by delivery of a CD7 RNP. In other examples, a CD7 RNP is delivered, which is followed by delivery of a TRAC RNP.

Sequential delivery, as with dual/multiplex delivery of cargos provides therapeutic advantages, wherein cells can be modified to possess multiple new features that enhance their ability to target tumor cells or to effectively kill the tumor cells.

In certain examples, sequential delivery of the exogenous cargo (e.g., at least two RNPs) provides a synergistic effect as compared to either sequential delivery and/or a control (an immune cell not including exogenous cargo). For example, the synergistic effect is an effect when two or more exogenous cargos are delivered (e.g., sequentially) to create an effect that is greater than either one of them by itself.

### T Cell Receptor α Constant (TRAC)

TRAC RNP was delivered and was followed, e.g., two days, later by delivery of the CD7 RNP.

Disruption of TCR expression after TRAC RNP delivery is measured by loss/reduction of cluster of differentiation 3 (CD3) expression, e.g., by detecting the presence of CD3 on the cell surface using standard methods such as flow cytometry. CD3 forms a surface complex with the TCR (T cell receptor); by staining the cells for the presence of CD3, the TRAC knockdown is detected. Disruption of cluster of differentiation 7 (CD7) expression after delivery of CD7 RNP is measured by loss/reduction of CD7 expression, e.g., by detecting the presence of CD7 on the cell surface using standard methods such as flow cytometry.

### Advantages of the Claimed Invention, e.g., Sequential RNP/RNP Delivery

Repeated transfection events in a given population of cells is challenging because transfection can cause stress to cells. Many transfection methods such as lipofection and electroporation are associated with cellular toxicity to varying degrees and while one transfection event can be tolerated, repeated transfection events can result in significantly reduced cell viability and high levels of cell death. Even those cells that survive can be damaged and unable to correctly process the exogenous cargo that has been delivered.

RNPs are gene editing tools that cut DNA such that the DNA is subsequently be repaired by the cell. The cell must be in a healthy state in order to carry out this repair process and remain viable at the same time. If the cell is damaged, the likelihood of a gene edit being successfully carried out is reduced. A transfection process that delivers an RNP to a cell must have low toxicity in order to yield cells that are viable and have the gene edit present.

The SOLUPORE® delivery system provides a gentle transfection process that enables efficient delivery of cargos into cells. When RNPs are delivered using the SOLUPORE® system, efficient gene editing is achieved with concomitant retention of high cell viability. The absence of cell stress is such that a second RNP transfection process can be carried out with equally high levels of edit efficiency and cell viability. This means that complex engineering of immune cells can be carried out for the purpose of cell therapy manufacture. Moreover, if the starting material is fragile, such as patient-derived cells such as T cells, NK cells or tumor infiltrating lymphocytes (TILs), it is important to use a gentle process for complex engineering such as the SOLUPORE® delivery system. Other starting material such as iPSC-derived T cells or NK cells are also fragile and so it is desirable to carry out repeat transfections in these cells using systems such as the SOLUPORE® delivery system, which will cause minimal stress to these cells.

### Definitions

The following definitions are included for the purpose of understanding the present subject matter and for constructing the appended patent claims. The abbreviations used herein have their conventional meanings within the chemical and biological arts.

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

“Patient” or “subject in need thereof” refers to a living member of the animal kingdom suffering from or who may suffer from the indicated disorder. In embodiments, the subject is a member of a species comprising individuals who may naturally suffer from the disease. In embodiments, the subject is a mammal. Non-limiting examples of mammals include rodents (e.g., mice and rats), primates (e.g., lemurs, bushbabies, monkeys, apes, and humans), rabbits, dogs (e.g., companion dogs, service dogs, or work dogs such as police dogs, military dogs, race dogs, or show dogs), horses (such as race horses and work horses), cats (e.g., domesticated cats), livestock (such as pigs, bovines, donkeys, mules, bison, goats, camels, and sheep), and deer. In embodiments, the subject is a human.

The terms “subject,” “patient,” “individual,” etc. are not intended to be limiting and can be generally interchanged. That is, an individual described as a “patient” does not necessarily have a given disease, but may be merely seeking medical advice.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

In the descriptions herein and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

As used herein, an “isolated” or “purified” nucleic acid molecule, polynucleotide, polypeptide, or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) or polypeptide is free of the amino acid sequences or nucleic acid sequences that flank it in its naturally-occurring state. Purified also defines a degree of sterility that is safe for administration to a human subject, e.g., lacking infectious or toxic agents

Relative to a control level, the level that is determined may an increased level. As used herein, the term “increased” with respect to level (e.g., cytokine release, gene regulation, or metabolic rate of T cells after the described SOLUPORE® methods) refers to any % increase above a control level. In various embodiments, the increased level may be at least or about a 5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, at least or about a 95% increase, relative to a control level.

Relative to a control level, the level that is determined may a decreased level. As used herein, the term “decreased” with respect to level (e.g., cytokine release, gene regulation, or metabolic rate of T cells after the described SOLUPORE® methods) refers to any % decrease below a control level. In various embodiments, the decreased level may be at least or about a 5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, at least or about a 95% decrease, relative to a control level.

The increase or decrease may also be expressed as fold-difference or log-difference. For example, Log base 2 (or log 2) was used to normalize the results along an axis with equal values for upregulated and downregulated genes. An exemplary calculation is shown below:

gene A treated vs control=7.0 (overexpressed);

gene B control vs treated=7.0 or treated vs control=0.142 (underexpressed).

Both are overexpressed or underexpressed with the same intensity but in a linear scale this is not reflected. Alternatively, gene A is 7.0 fold up and gene 2 is 0.142 down regulated. If this is expressed in log₂ then gene A is 2.81 fold upregulated and gene B is −2.81 fold downregulated.

### EXAMPLES

The following examples illustrate certain specific embodiments of the invention and are not meant to limit the scope of the invention.

Embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

### Example 1: Efficient Engineering of Primary Human T Cells for Ex Vivo Cell Therapy Applications Using the SOLUPORE® Delivery Method

Efficient and Versatile Engineering of Primary Human T Cells

Next generation immune cell therapies will require complex editing that may include both addition and deletion of cell functions. Therefore, the ability of the SOLUPORE® delivery system was used to both introduce functionality to T cells, using mRNA, and to delete functionality, using CRISPR-Cas9 protein-gRNA ribonucleoprotein (RNP) complexes. To further examine the versatility of the platform, these cargos were delivered in combination or sequentially.

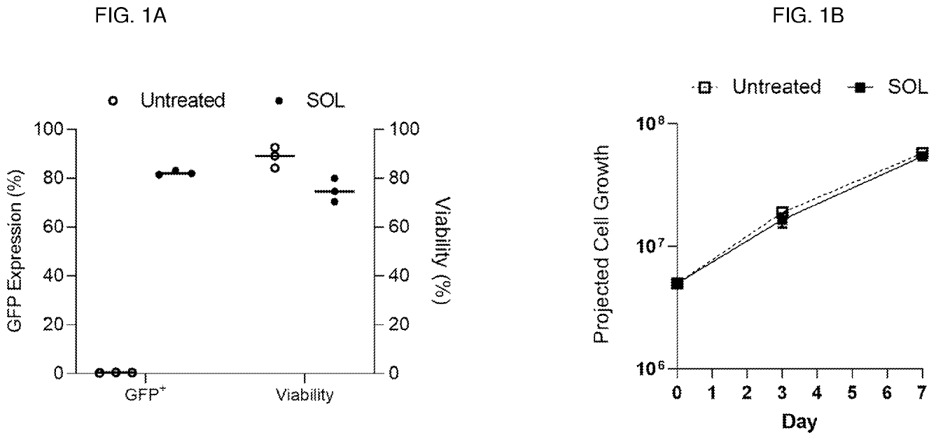

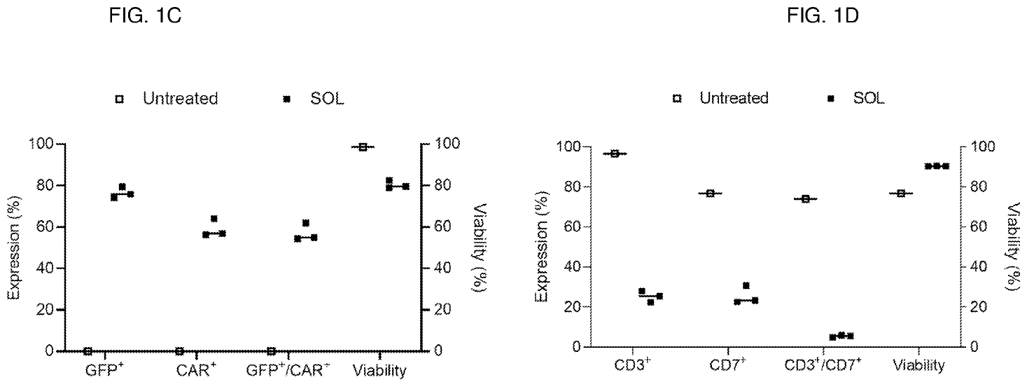

GFP mRNA was delivered to T cells from three human donors and GFP expression and cell viability were examined at 24 hr post-transfection. GFP expression was greater than 80% in each donor with high cell viability retained (FIG. 1A). The proliferation of T cells following delivery of GFP mRNA remained similar to that of untreated cells (FIG. 1B).

More complex cargo delivery scenarios were further examined. When CAR mRNA was co-delivered with GFP mRNA, more than 57% of the population was GFP⁺/CAR⁺ at 24 hr post-transfection, again with high cell viability retained (FIG. 1C). CRISPR-Cas9 RNP complexes targeting the TRAC and CD7 genes were delivered to T cells individually and sequentially. When delivered individually, CD3 and CD7 expression in the treated populations was reduced to approximately 25% in both cases (FIG. 1D). When the TRAC RNP was delivered and was followed two days later by the CD7 RNP, approximately 5% of the treated population retained a CD3⁺/CD7⁺ phenotype and viability remained high when examined 4 days post-delivery of the CD7 RNP (FIG. 1D).

The success of the seminal CAR T ‘living’ drugs was achieved in spite of complex manufacturing and logistical processes that have created a new paradigm for drug manufacture (Freitag F, Maucher M, Riester Z, Hudecek M. New targets and technologies for CAR-T cells. Curr Opin Oncol 2020; 32:510-517). However, hard lessons have already been learned about the cost and complexity of the manufacturing process where one batch is manufactured for one patient and the quality of the patient-derived material (Namuduri M, Brentjens R J. Enhancing CAR T cell efficacy: the next step toward a clinical revolution? Expert Rev Hematol 2020; 13:533-543). It is now widely accepted that future key focus areas must include optimization of cell therapies for liquid tumors, acceleration of the innovation cycle time to enable success in solid tumors and transformation of manufacturing processes. It is predicted that virus-free protocols could play a key role in all of these aspects, ultimately improving patient access (Freitag F, Maucher M, Riester Z, Hudecek M. New targets and technologies for CAR-T cells. Curr Opin Oncol 2020; 32:510-517). In addition, continuing advances in gene engineering tools mean that genome targeting is now possible with non-viral approaches (Roth T et al. Reprogramming human T cell function and specificity with non-viral genome targeting. Nature 2018; 559:405-9).

The SOLUPORE® delivery system was developed as an advanced technology aimed at addressing development and manufacturing needs of the cell therapy field. Described herein, the delivery efficiency of the system with a range of cargo types was demonstrated. The ability of the platform to support multiplex delivery and sequential gene edits without compromising cell viability is an important feature. If targeting and efficacy are to be enhanced in autologous cell therapies for both liquid and solid tumors, cells will require multiple modifications using steps that can integrate with manufacturing processes. This may involve multiplex or sequential engineering steps. Similar demands apply to allogeneic approaches where cell rejection and GvHD mean that complex editing is likely to be required.

Limitations in viral vector capacity and electroporation toxicity mean that these modalities may be unsuitable for many complex engineering regimes. Moreover, the long lead time required to design and generate even research grade viral vectors means that timelines may be longer than desired at the development stage. This is of particular concern in relation to progressing novel approaches for solid tumors where targeting and efficacy challenges mean that large numbers of candidate target antigens and cell potency enhancements will need to be tested. It will be necessary to evaluate a myriad of cell compositions in a rapid, high-throughput fashion that is likely to be highly constrained if wholly reliant on viral vectors. Thus new non-viral intracellular delivery modalities are needed. The studies reported here show that the system is compatible with optimization of CAR T for liquid tumors and the acceleration of the innovation cycle time required for impactful progress in tackling solid tumors.

Cell viability must be maintained if cell engineering is to be useful. There is interest in the field in developing alternative non-viral delivery methods that can be efficient whilst also being gentle on cells. The studies reported here demonstrate that the transfection process has a minimal impact on cell viability.

In summary, the SOLUPORE® delivery system represents an attractive non-viral delivery platform that efficiently engineers T cells while retaining high levels of cell viability and so is well-suited to address the manufacturing challenges for next generation cell therapy products

Materials and Methods Used Herein

Cell Isolation and Culture

PBMC were isolated from fresh leukopaks using lymphoprep density gradient medium (StemCell) and cryopreserved using standard methods. Upon thaw, PBMC were initiated to T cells using soluble CD3 (clone OKT3) and CD28 (clone 15E8) antibodies (both Miltenyi Biotech), each at 100 ng/ml. Cells were initiated for 3 days in complete culture media consisting of CTS OpTimizer+supplement (Gibco) with 5% Physiologix serum replacement (Nucleus Biologics), 1% L-Glutamine and 250 IU/ml IL-2 (CellGenix).

Transfection Using the SOLUPORE® Delivery System

The transfection method was adapted from that previously described (O'Dea S, Annibaldi V, Gallagher L, Mulholland J, Molloy E, Breen C, Gilbert J, Martin D, Maguire M, Curry F. Vector-free intracellular delivery by reversible permeabilization. PLoS ONE 2017; 12). Cells were transferred to either 96-well filter bottom plates (Agilent) at 3.5×10⁵ cells per well or to transfection pods (Avectas) at 6×10⁶ cells per pod. Culture medium was removed from the 96-well plates by centrifugation at 350×g for 120 sec and from the pods by gravity flow. Cargos were combined with delivery solution (32.5 mM sucrose, 106 mM potassium chloride, 5 mM Hepes in water) and 1 μl or 50 μl was delivered onto the cells in the 96-well plates or pods respectively. For delivery of RNP and mRNA, delivery solution also contained 10% and 12% v/v ethanol respectively. Following a 30 sec incubation at room temperature, 50-2000 μl 0.5× phosphate buffered saline solution (68.4 mM sodium chloride, 1.3 mM potassium chloride, 4.0 mM sodium hydrogen phosphate, 0.7 mM potassium dihydrogenphosphate) was added and after 30 sec, complete culture medium was added.

Delivery of GFP mRNA, CAR mRNA and RNP Complexes

GFP mRNA and CD19 CAR mRNA (both TriLink Biotechnologies) were delivered to a final concentration of 2 μg and 3.3 μg/1×10⁶ cells for the SOLUPORE® delivery system and for electroporation respectively. CD19 CAR expression was evaluated using a biotin-conjugated CD19 CAR detection reagent (Miltenyi Biotec) followed by Streptavidin-PE with 7AAD as a viability stain. Cas9 protein (Integrated DNA Technologies) was delivered at a final concentration of 3.3 μg/1×10⁶ cells and precomplexed with a 2 molar excess of gRNA (Cas9—2.48 μM and gRNA 4.96 μM; Integrated DNA Technologies). The sequence for human TRAC-targeting gRNA was AGAGTCTCTCAGCTGGTACA (SEQ ID NO: 1) and for human CD7-targeting gRNA was GGAGCAGGTGATGTTGACGG (SEQ ID NO: 2). CD3 and CD7 (antibody clone CD7-6B7, BioLegend) expression were analysed by flow cytometry.

Proliferation Assay

Following transfection with GFP mRNA, T cells were counted using an NC-Slide A8™ and Solution 13 (ChemoMetec) on the NC-3000™ according to the manufacturer's protocol. Samples were adjusted to a viable cell density of 1×10⁶ cells/mL and 200 μL was transferred to a u-bottom 96-well plate (Greiner). Cells were placed in a humidified 37° C., 5% CO₂ incubator for 72 h. Samples were then counted, re-seeded at a viable cell density of 1×10⁶ cells/mL in fresh medium and incubated for a further 96 h. Projected cell growth over 7 days was calculated by multiplying a starting cell number of 5×10⁶ viable cells by the observed fold growth over 3 days, this value was then multiplied by the observed fold growth over following 4 days.

Flow Cytometry Analysis

Flow cytometry was performed using NovoCyte 3000. Data were examined using NovoExpress software (Acea Biosciences).

Statistics

A Wilcoxon matched pairs signed rank test was carried out on the cell proliferation data.

### Example 2: Multiple Simultaneous Edits on SOLUPORE® Delivery Systems

The SOLUPORE® delivery system was used to assess the editing of multiple target sites following simultaneous delivery of multiple Cas9 RNPs. Both the SOLUPORE® Research Platform delivery system and clinical platform (SUS) were used in these experiments. Isolated CD3+ cells from 3 healthy donors were cultured in TexMACS (Miltenyi Biotech) 5% HAB (Valley Biomedical) and activated with TransAct (TA; Miltenyi Biotech) and 120/mL IL-2 (Cell Genix) for 3 days. CRISPR Cas9 RNPs targeting the TRAC (guide RNA sequence AGAGTCTCTCAGCTGGTACA (SEQ ID NO: 1)), CD7 (guide RNA sequence GGAGCAGGTGATGTTGACGG (SEQ ID NO: 2)) and 3-2 microglobulin (B2m; guide RNA sequence GGCCGAGATGTCTCGCTCCG (SEQ ID NO: 3)) locus were prepared (2:1 guide RNA to Cas9 molar ratio) and delivered (3 μg per 1×10⁶ cells) to the activated cells (the SOLUPORE® Research Platform delivery system at 6×10⁶ cells per replicate and 20×10⁶ cells per replicate for the clinical platform).

Post-delivery, cells were cultured for 4 days at 37° C., 5% CO₂ and 95% humidity (split on day 2) and then analyzed for edit via protein expression. Cells were stained with antibodies against CD3 (TRAC RNP, antibody clone SK7, BioLegend), CD7 (CD7 RNP; CD7-SB7, BioLegend) and HLA (B2m RNP; antibody clone W6/32, BioLegend) and expression was assessed using flow cytometry. For B2M KO, the antibody used was anti-HLA-APC measuring HLA-ve response, as MHC-1 has human leukocyte antigen (HLA)-encoded alpha chain that binds the peptide and a Beta-2-microglobulin (B2M) protein that acts as a stabilizing scaffold and B2M gene knockout results in loss in surface expression of HLA-1.

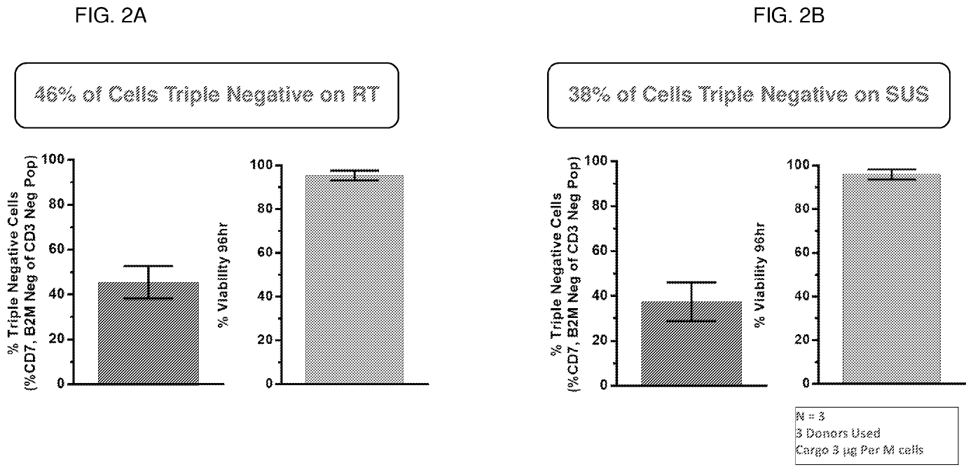

The percentage population that was negative for all 3 proteins was 46±7 and 38±9 from SOLUPORE® Research Platform delivery system and the clinical use platform, respectively (3 donors, 3 technical repeats); see FIGS. 2A and 2B, respectively. Comparable performance on the SOLUPORE® Research Platform delivery system and the clinical platform was observed, with 10% or less triple edits by other techniques.

Effect of Alcohol (Ethanol) on RNP-Edit Efficiency Post-Delivery by the SOLUPORE® Delivery Systems

Experiments were performed to determine to whether ethanol had an effect on RNP-edit efficiency post-delivery using the SOLUPORE® delivery system. Additionally, the experiments were performed to ascertain an optimal ethanol concentration for editing following delivery of RNP by the SOLUPORE® delivery system, for example, the maximum ethanol concentration to allow for optimal Cas9-induced edit was determined. An increase in ethanol may allow greater amount of cargo delivery to the cell allowing for greater edit efficiency.

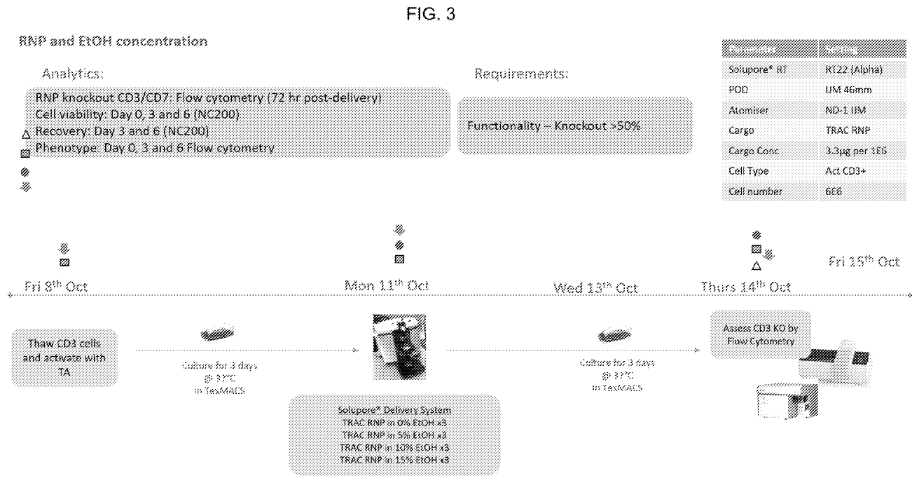

Cas9 RNP—TRAC sgRNA was prepared at 2:1 ratio at 0.4 μg/μL (equiv to 3.3 μg per 1×10⁶ cells); S Buffer (32.5 mM sucrose; 106 mM potassium chloride; 5 mM HEPES) solutions were prepared with 0, 5, 10 and 15% ethanol with RNP and the experiments were carried out on the SOLUPORE® delivery system with the S buffer solutions at each ethanol concentration. The experimental design is shown in FIG. 3.

“S Buffer” includes a hypotonic physiological buffered solution (78 mM sucrose, 30 mM KCl, 30 mM potassium acetate, 12 mM HEPES) for 5 min at 4° C. (Medepalli K. et al., Nanotechnology 2013; 24(20); incorporated herein by reference in its entirety). In some examples, potassium acetate is replaced with ammonium acetate in the S Buffer. S buffer is further described in international application WO 2016/065341, e.g., at ¶ [0228]-[0229] and incorporated herein by reference in its entirety. For example, the S buffer used in this series of experiments included 32.5 mM sucrose; 106 mM potassium chloride; and 5 mM HEPES.

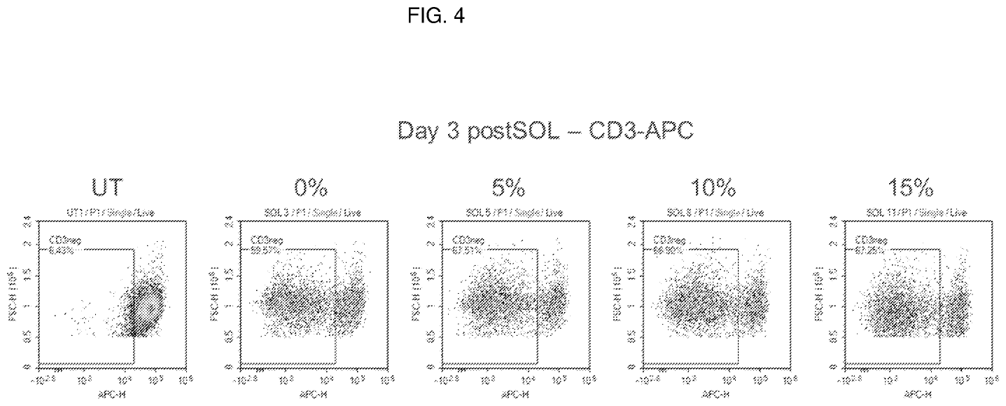

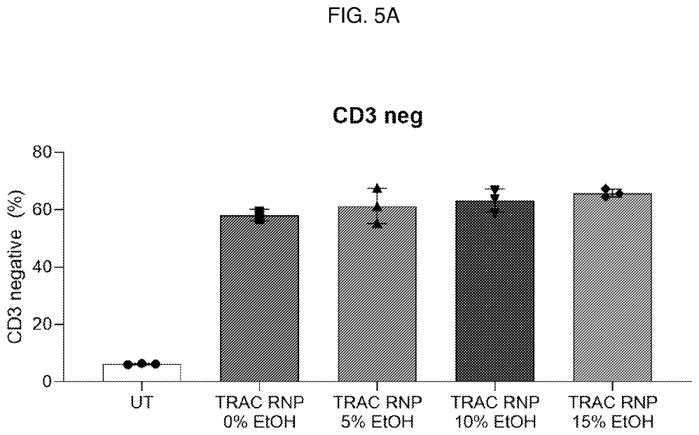

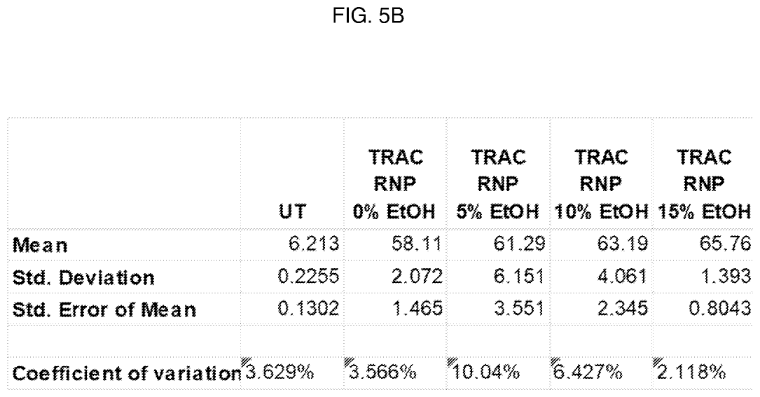

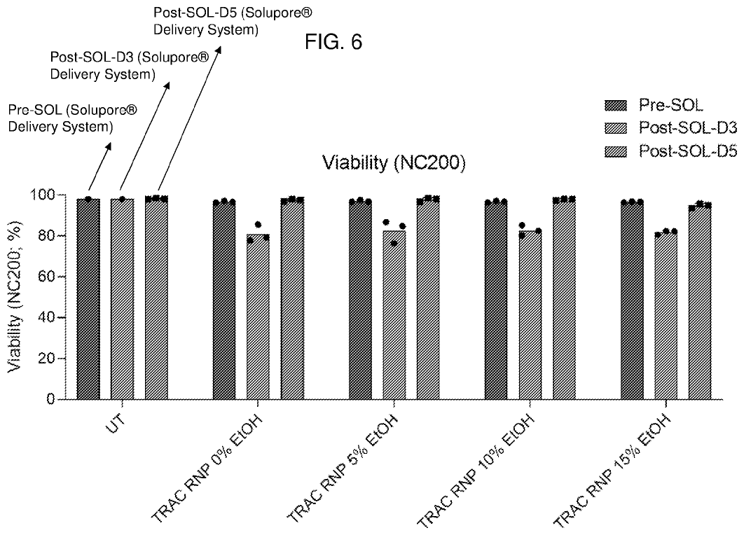

Conclusion: CD3 edit efficiencies (e.g., monitoring TRAC RNP) at each ethanol concentration was tested post-delivery using the SOLUPORE® Delivery System. See FIG. 4 depicting representative flow cytometry plots from cells stained with an antibody targeting CD3 (gated off the live population) and FIG. 5A shows a bar graph showing that the level of CD3 edit increased modestly with increasing concentrations of ethanol (0% EtOH-58% to 15% EtOH-66%), and the results are further summarized in the table in FIG. 5B.

### Example 3: CRISPR/RNP Editing in Human Primary T Cells

Three different experiments were carried out whereby the cargo was delivered for a single edit and cargos were administered simultaneously.

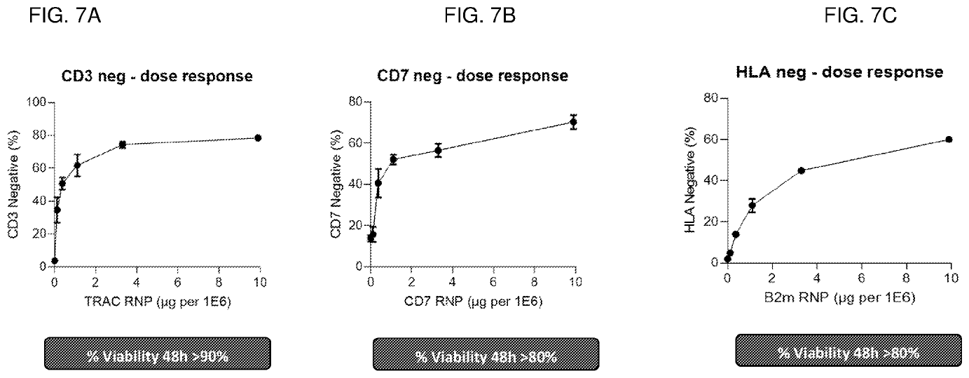

CRISPR/RNP editing was evaluated in human primary T cells. CRISPR/RNPs single edits at multiple target sites were assessed and the edit efficiencies ranged from 60-80%, and cell viability was 80-90%. See FIGS. 7A-7C. Successful edits, across a range of RNP concentrations (0.12; 0.37; 1.1; 3.3 and 9.9 μg per 1×10⁶ cells), for TRAC (guide RNA sequence AGAGTCTCTCAGCTGGTACA (SEQ ID NO: 1); CD3), CD7 (guide RNA sequence GGAGCAGGTGATGTTGACGG (SEQ ID NO: 2)), and B2M (guide RNA sequence GGCCGAGATGTCTCGCTCCG (SEQ ID NO: 3); HLA) RNPs with >80% viability was observed.

T Cell Phenotype was Preserved During Editing

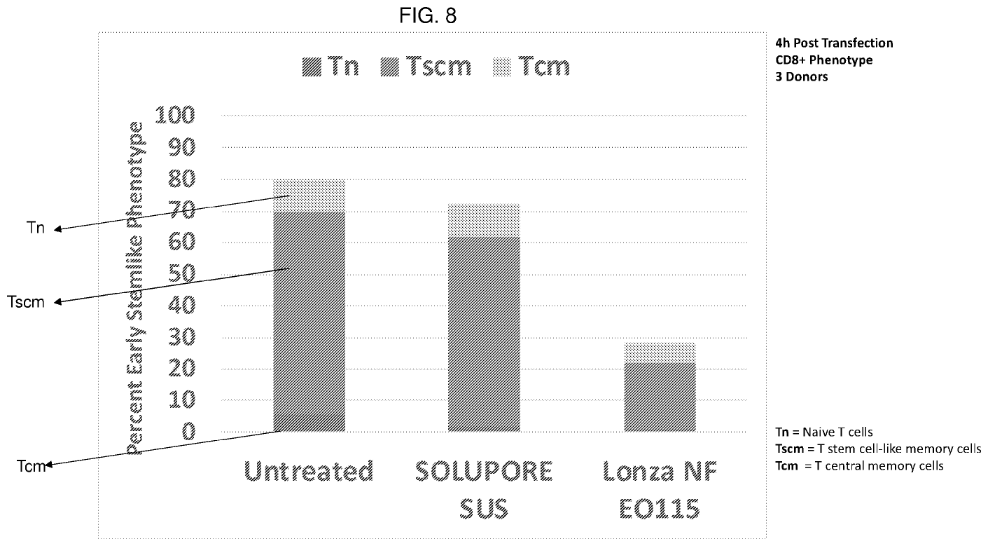

Gene editing is evident within 4 hours post RNP delivery, so preservation of stemlike cell phenotype at early time points was critical. CD8+ cells were the main cytotoxic T cells and similar trends were seen with CD4+ Helper T cells. See FIG. 8 (where a 4 hour timepoint was recorded).

The cells using the SOLUPORE® Delivery System showed a higher percent stemlike phenotype than nucleofection (NF; Lonza Nucleofection) during the gene editing window. For example, the early time-point window may include 3 hours (see, e.g., Kim et al. Highly efficient RNA-guided genome editing in human cells via delivery of purified Cas9 ribonucleoproteins. Genome Res. 2014; 24(6):1012-1019, incorporated herein by reference in its entirety), or a late time-point window may be where gene editing is complete by 24 hours (see, e.g., Brinkman E K, et al. Kinetics and Fidelity of the Repair of Cas9-Induced Double-Strand DNA Breaks. Mol Cell. 2018; 70(5):801-813.e6; incorporated herein by reference in its entirety).

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All references, e.g., U.S. patents, U.S. patent application publications, PCT patent applications designating the U.S., published foreign patents and patent applications cited herein are incorporated herein by reference in their entireties. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

While this invention has been particularly shown and described with references to preferred embodiments thereof, it will be understood by those skilled in the art that various changes in form and details may be made therein without departing from the scope of the invention encompassed by the appended claims.

1. An immune cell comprising at least two exogenous cargos, wherein the exogenous cargo comprises a ribonucleoprotein (RNP), a nucleic acid, a protein, or any combination thereof.

2. The immune cell of claim 1, wherein the RNP comprises a T cell receptor alpha constant (TRAC) RNP or cluster of differentiation 7 (CD7) RNP.

3. The immune cell of claim 1, wherein the at least two exogenous cargos are sequentially delivered.

4. The immune cell of claim 1, wherein viability of the immune cell is increased compared to a control immune cell.

5. A method of delivering at least two exogenous cargos across a plasma membrane of a non-adherent immune cell, comprising,
providing a population of non-adherent cells; and
contacting the population of cells with a volume of an isotonic aqueous solution, the aqueous solution including the exogenous cargo and an alcohol at greater than 0.2 percent (v/v) concentration.

6. The method of claim 5, wherein the at least two exogenous cargos are sequentially delivered.

7. The method of claim 5, wherein the at least two exogenous cargos comprise a ribonucleoprotein (RNP), a nucleic acid, a protein, or any combination thereof.
